(19) United States
(12) Patent Application Publication
(10) Pub. No.: US 2022/0370824 A1
(43) Pub. Date: Nov. 24, 2022

(54) LIGHT SUPPLY METHOD AND LIGHT SUPPLY SYSTEM FOR PHOTOTHERAPY

(71) Applicant: National Taiwan University of Science and Technology, Taipei (TW)

(72) Inventors: Chien-Yu Chen, Taipei (TW); Hung-Wei Chen, Taipei (TW)

(73) Assignee: National Taiwan University of Science and Technology, Taipei (TW)

(21) Appl. No.: 17/523,944

(22) Filed: Nov. 11, 2021

(30) Foreign Application Priority Data
May 21, 2021 (TW) 110118436

(51) Int. Cl. A61N 5/06

(52) U.S. Cl. CPC A61N 5/0618; A61N 2005/065; A61N 2005/0664; A61N 2005/0626; A61N 2005/0663

(57) ABSTRACT

A light supply method and a light supply system for phototherapy are provided. The light supply method includes the following. A plurality of light emitting modules of a light source device are driven so that a light source device outputs a first light. The first light is sensed by a light sensing module. A light parameter corresponding to best physiology of a user is received. A light output ratio of the light emitting modules is adjusted based on the light parameter, thereby adjusting the first light to a second light. The light emitting modules respectively have different central wavelengths. A half-height width of a plurality of spectra of the light emitting modules is less than 30 nanometers.

### CROSS-REFERENCE TO RELATED APPLICATION

This application claims the priority benefit of Taiwan application serial no. 110118436, filed on May 21, 2021. The entirety of the above-mentioned patent application is hereby incorporated by reference herein and made a part of this specification.

### BACKGROUND

### Technical Field

The disclosure relates to a light supply method and a light supply system, and in particular to a light supply method and a light supply system used for phototherapy.

### Description of Related Art

At present, many long-term care institutions provide long-term care for care recipients with brain diseases (such as dementia, depression). However, due to lack of human resources for providing long-term care, most nursing homes adopt indoor semi-centralized care, and the care recipients mostly stay in indoor care settings. The care recipients are prone to emotional problems and insomnia due to factors such as insufficient indoor lighting and little exposure to outdoor sunlight. Literature has pointed out that most of the long-term bedridden elderly are prone to an abnormal circadian rhythm, which leads to sleepiness in the daytime and lack of sleep at night. Therefore, about 50 to 80% of care recipients in long-term care institutions now rely on at least one medication for sleep, such as medication to treat Alzheimer's disease (Cholinesterase inhibitors, NMDA receptor antagonists), antipsychotic medication, antidepressants, etc., in order to alleviate the symptoms that the care recipients might have. However, the above methods may cause side effects such as sleepiness, constipation, low blood pressure, tremor, stiffness of the body or limbs, and poor balance. Therefore, normalizing the routine of the care recipients and slowing down the deterioration of symptoms is one of the issues that those skilled in the art are focused on.

### SUMMARY

The disclosure provides a light supply method and a light supply system that provide phototherapy.

A light supply method of the disclosure is used for phototherapy. The light supply method includes the following. Multiple light emitting modules of a light source device are driven so that the light source device outputs a first light. A light sensing device senses the first light to receive a first light parameter of the first light. A second light parameter corresponding to best physiology of a user is received. A light output ratio of the light emitting modules is adjusted based on the second light parameter, and the first light parameter is adjusted to the second light parameter, so that the first light is adjusted to a second light. The light emitting modules respectively have a different central wavelength. A half-height width of multiple spectra of the light emitting modules is less than 30 nanometers.

A light supply system of the disclosure is used for phototherapy. The light supply system includes a light source device, a light sensing device, and a monitoring module. The light source device includes multiple light emitting modules. The light emitting modules are driven to provide a first light. The light sensing device is configured to sense the first light to receive a first light parameter of the first light. The monitoring module is coupled to the light source device and the light sensing device. The monitoring module is configured to receive a second light parameter corresponding to best physiology of a user, and adjust a light output ratio of the light emitting modules based on the second light parameter and adjust the first light parameter to the second light parameter, thereby adjusting the first light to a second light. The light emitting modules respectively have a different central wavelength. A half-height width of multiple spectra of the light emitting modules is less than 30 nanometers.

Based on the above, multiple light emitting modules of the light source device of the disclosure have different central wavelengths, and the half-height width of multiple spectra of the light emitting modules is lower than 30 nanometers. Therefore, the light supply method and the light supply system of the disclosure accurately provide the second light with the second light parameter, so that the physiology of the user is improved.

To provide a further understanding of the above features and advantages of the disclosure, embodiments accompanied with drawings are described below in details.

### BRIEF DESCRIPTION OF THE DRAWINGS

FIG. 1 illustrates a schematic diagram of a light supply system according to an embodiment of the disclosure.

FIG. 2 illustrates a flow chart of a light supply method according to an embodiment of the disclosure.

FIG. 3 illustrates a schematic diagram of the operation of a light sensing device according to an embodiment of the disclosure.

FIG. 4A illustrates a schematic diagram of the operation of a light sensing device according to another embodiment of the disclosure.

FIG. 4B illustrates a schematic diagram of sensing and circadian stimulus results according to the operation of FIG. 4A.

FIG. 5 illustrates a schematic diagram of an operational interface according to an embodiment of the disclosure.

FIG. 6 illustrates a schematic diagram of an operational interface according to another embodiment of the disclosure.

### DESCRIPTION OF THE EMBODIMENTS

A portion of the embodiments of the disclosure will be described in detail in conjunction with the accompanying drawings. In the following description, the same element symbols appear in different drawings will be regarded as the same or similar elements. These embodiments are only part of the disclosure and do not disclose all the implementation methods of the disclosure. Specifically, these embodiments are just examples of the device and method in the claims of the disclosure.

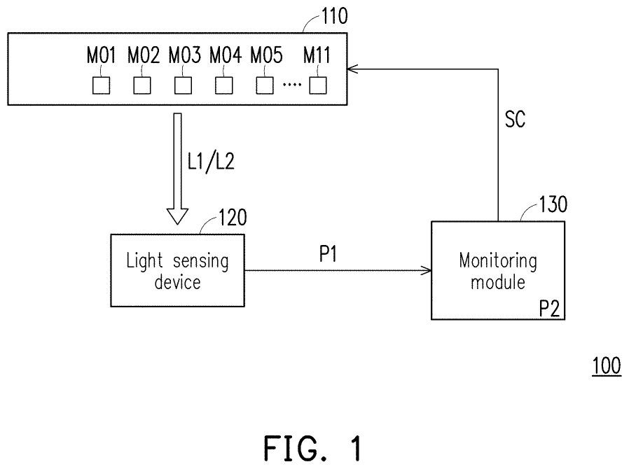

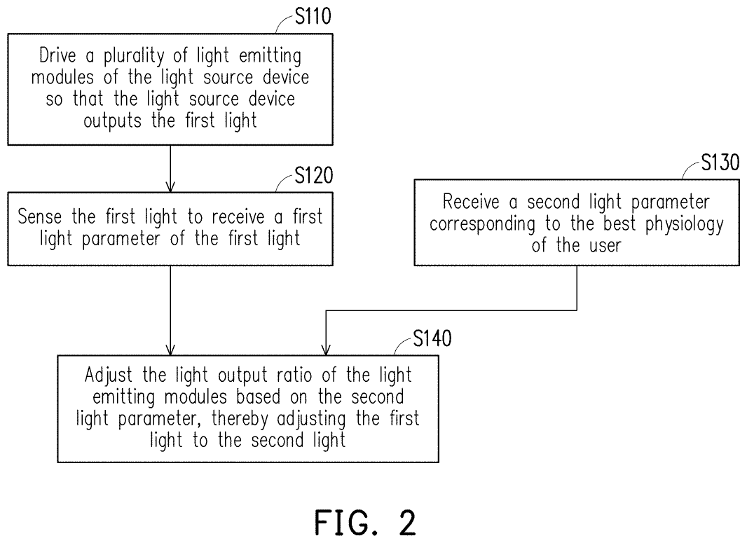

Referring to FIGS. 1 and 2, FIG. 1 illustrates a schematic diagram of a light supply system according to an embodiment of the disclosure. FIG. 2 illustrates a flow chart of a light supply method according to an embodiment of the disclosure. In this embodiment, a light supply system 100 may provide a first light L1 and a second light L2 to a care setting. The light supply system 100 includes a light source device 110, a light sensing device 120, and a monitoring module 130. In step S110, the light source device 110 is driven to provide the first light L1. In this embodiment, the first light L1 may be an initial light (for example, a predetermined output light during startup). In this embodiment, the light source device 110 includes 11 sets of light emitting modules M01 to M11 (the disclosure is not limited to the number of light emitting modules M01 to M11). The light emitting modules M01 to M11 have different central wavelengths. For example, the central wavelengths of the light emitting modules M01 to M11 are shown in Table 1 (the disclosure is not limited to the central wavelengths of Table 1).

TABLE 1

| Light emitting module | Central wavelength (nanometer) |
|---|---|
| M01 | 425 |
| M02 | 450 |
| M03 | 475 |
| M04 | 505 |
| M05 | 525 |
| M06 | 540 |
| M07 | 555 |
| M08 | 595 |
| M09 | 610 |
| M10 | 635 |
| M11 | 660 |

The light emitting modules M01 to M11 each include at least one light emitting device. In this embodiment, the light emitting modules M01 to M11 each include at least one light emitting device. In this embodiment, the light emitting device may be realized by a high-brightness light emitting diode. In addition, the light emitting modules M01 to M11 are further designed so that the half-height width of a plurality of spectra of the light emitting modules M01 to M11 is less than 30 nanometers. In this embodiment, at least one light emitting device of the light emitting modules M01 to M11 is configured in an interleaved manner with each other. In addition, among the light emitting modules M01 to M11, the difference between the central wavelengths of two light emitting modules with the closest central wavelengths is less than or equal to 40 nanometers. For example, in Table 1, the wavelength difference between the central wavelength of the light emitting module M01 and the central wavelength of the light emitting module M02 is 25 nanometers. The wavelength difference between the central wavelength of the light emitting module M02 and the central wavelength of the light emitting module M03 is 25 nanometers, and so on.

In step S120, the light sensing device 120 receives the first light L1 to receive a first light parameter P1. In this embodiment, the light sensing device 120 may be a set of devices formed by at least one light sensor. In this embodiment, the light parameter may be a parameter such as illuminance and intensity spectrum provided by the output light. Therefore, the first light parameter P1 may be parameters including illuminance, spectrum, color temperature provided by the first light L1. In this embodiment, the light sensing accuracy of the light sensing device 120 is 1 to 10 nanometers. Therefore, the light sensing device 120 may accurately receive the first light parameter P1.

In this embodiment, the monitoring module 130 is coupled to the light source device 110 and the light sensing device 120. In step S130, the monitoring module 130 receives a second light parameter P2 corresponding to the best physiology of the user (for example, the care recipient). The best physiology is data associated with at least one of the user's sleep quality, melatonin inhibition during the daytime, and a depression scale (for example, CES-D) analysis. Therefore, the best physiology is the best data associated with at least one of the user's sleep quality, melatonin inhibition during the daytime, and the depression scale analysis. For example, the best physiology may be the best data (or target data) of reduced psychotic behaviors, delayed deterioration of conditions, or improved sleep quality for patients with mild to moderate dementia. The best data may be received in advance from at least one of a sleep bracelet, a saliva test (to receive the melatonin content), and CES-D.

In step S140, the monitoring module 130 adjusts the light output ratio of the light emitting modules M01 to M11 based on the second light parameter P2. In this way, the monitoring module 130 adjusts the first light L1 to the second light L2. For example, the monitoring module 130 stores the second light parameter P2 corresponding to the best physiology. When phototherapy is performed, the monitoring module 130 may provide a control signal SC to control the light source device 110 to adjust the first light L1 to the second light L2. That is, the monitoring module 130 adjusts the first light parameter P1 to the second light parameter P2 and provides the control signal SC accordingly. Therefore, the light source device 110 outputs the second light L2 in response to the control signal SC.

It is worth mentioning that the light emitting modules M01 to M11 have different central wavelengths. The light emitting modules M01 to M11 are further designed so that the half-height width of a plurality of spectra of the light emitting modules M01 to M11 is less than 30 nanometers. Therefore, through the method in this embodiment, the second light L2 with the second light parameter P2 may be accurately provided, so that the second light L2 may be used for phototherapy on the user. In this way, the light source device 110 may be controlled to accurately provide the second light L2, thereby improving the user's physiology. In addition, the light supply system 100 may meet CIE S026 and specifications and standards of measuring circadian stimulus (CS) values.

In this embodiment, the monitoring module 130 may be disposed outside of the light source device 110 and the light sensing device 120. The monitoring module 130 may be an electronic device with computing capabilities, such as a host or a server in any form. In some embodiments, the monitoring module 130 may be disposed inside the light source device 110 or the light sensing device 120. The monitoring module 130 may be a central processing unit (CPU), or other programmable general-purpose or special-purpose device such as a microprocessor, a digital signal processor (DSP), a programmable controller, application specific integrated circuits (ASIC), a programmable logic device (PLD), or other similar devices or a combination of the devices as described above, which may load and execute computer programs.

In this embodiment, the second light parameter P2 adapted for phototherapy may be one of the three sets of parameters shown in Table 2.

TABLE 2

| Second light parameter | Illuminance (lux) | Color temperature (k) |
|---|---|---|
| Parameter A | 6000 ± 200 | 5000 ± 200 |
| Parameter B | 3000 ± 200 | 5000 ± 200 |
| Parameter C | 3000 ± 200 | 8000 ± 200 |

In this embodiment, in a care setting, phototherapy is performed in a time period during the daytime (for example, during lunchtime). Specifically, according to the applicant's long-term experiments and statistics, performing the parameters A and B during lunchtime may significantly improve the sleep efficiency of the user at bedtime. The parameters A and C may further effectively inhibit the secretion of melatonin during the daytime. In this way, the parameters A and C help restore the user's circadian rhythm. In addition, the parameters A to C may significantly alleviate the user's depressive mood.

In some embodiments, when phototherapy is performed, in order to ensure that there is no light other than the second light L2 in the care setting, other light sources in the care setting are turned off or isolated.

In some embodiments, the light sensing device 120 may be calibrated. For example, before the light sensing device 120 is activated, the existing care setting is disposed as a dark room. Therefore, the light sensing device 120 may be activated in a dark room to perform dark calibration.

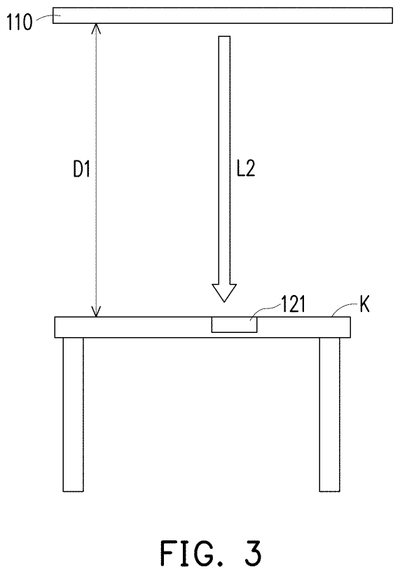

For example, FIGS. 1 and 3 may be referred to for the implementation details of the light sensing device. FIG. 3 illustrates a schematic diagram of the operation of a light sensing device according to an embodiment of the disclosure. In this embodiment, the light sensing device 120 includes a first light sensor 121. The first light sensor 121 and the light source device 110 are separated by a first predetermined distance D1. In this embodiment, the light source device 110 is disposed on the ceiling, and the first predetermined distance D1 is roughly the distance (for example, 140 cm) between a table top K and the ceiling. Therefore, the first light sensor 121 receives the second light L2 based on the first predetermined distance D1 to receive a first output light parameter of the second light L2. Therefore, the second light parameter P2 may correspond to the illuminance, spectrum, and color temperature of the second light L2 irradiating to the table top K. Generally, when the user (such as the care recipient) in the care setting participate in activities, they usually sit on a chair or wheelchair and participate in activities (such as dining or playing games) on the table top K. From the above, the light parameter provided to the table top K is an important parameter to maintain the quality of phototherapy. In addition, the monitoring module 130 may further monitor the first output light parameter. When the first output light parameter of the second light L2 deviates from the second light parameter P2 by more than a predetermined value (such as an predetermined intensity value), the monitoring module 130 adjusts the second light L2 through the control signal SC controlling the light source device 110, so that the first output light parameter of the second light L2 is approximate to the second light parameter P2 (such as one of the parameters A to C in Table 2). In this embodiment, the first light sensor 121 may be, for example, a CL500 illuminance meter or a CL-210 illuminance meter.

In some embodiments, the overall environment of the care setting needs to be considered. For example, the space of the care setting is limited. For example, white reflective walls or curtains limit the care setting to a space of 300 centimeters in length, 420 centimeters in width, and 210 centimeters in height in order to improve the adjustment accuracy of the second light L2.

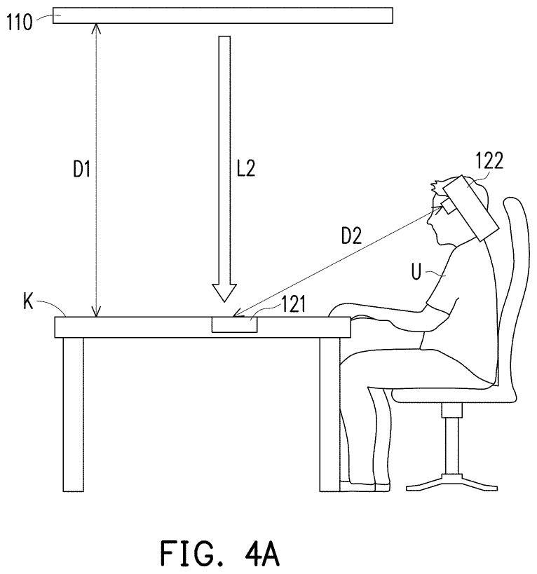

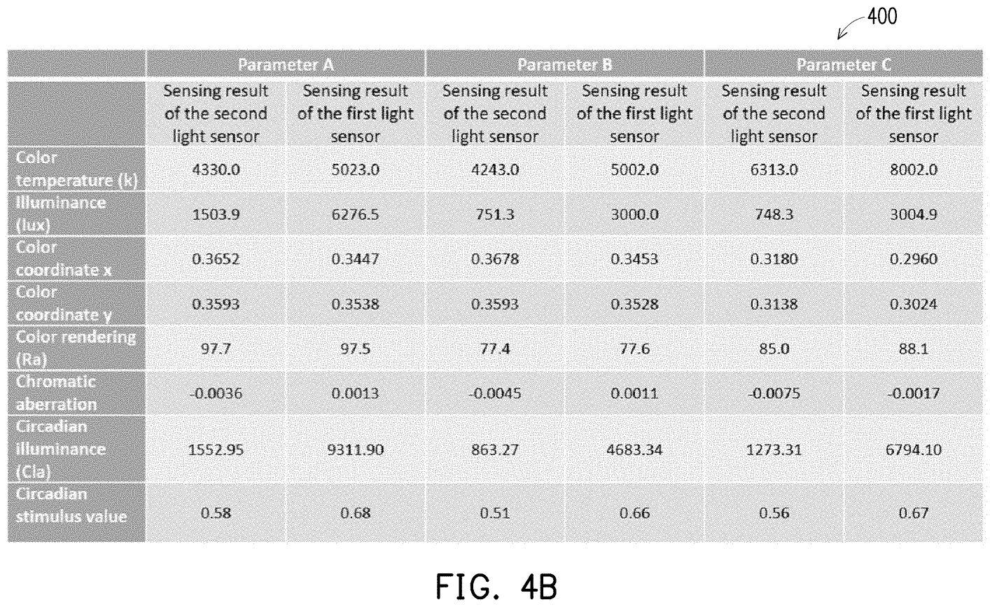

For example, FIGS. 1, 4A, and 4B may be referred to for the implementation details of the light sensing device. FIG. 4A illustrates a schematic diagram of the operation of a light sensing device according to another embodiment of the disclosure. FIG. 4B illustrates a schematic diagram of sensing and circadian stimulus results according to the operation of FIG. 4A. In this embodiment, the light sensing device 120 includes the first light sensor 121 and a second light sensor 122. Sufficient teaching for the implementation details of the first light sensor 121 may be received in the embodiment of FIGS. 1 and 3, so the details will not be repeated herein. In this embodiment, the second light sensor 122 and the first light sensor 121 are separated by a second predetermined distance D2. The second predetermined distance D2 is roughly the distance (for example, 40±5 centimeters) between the table top K and the face of a user U. Therefore, the first light sensor 121 receives the first output light parameter from the table top K. The second light sensor 122 receives a second output light parameter. The second output light parameter is roughly associated with the light parameter received by the user U's eyes. In this embodiment, the first light sensor 121 and the second light sensor 122 may be CL500 illuminance meters or CL-210 illuminance meters, for example. A sensing result 400 lists the sensing results and circadian stimulus results of the first light sensor 121 and the second light sensor 122 responding to the parameters A, B, and C listed in Table 2. In this embodiment, the first light sensor 121 may receive a plurality of parameters (for example, color temperature, illuminance, color coordinates, and color rendering circadian illuminance (Cla) from the table top K) of the parameters A, B, and C and the corresponding circadian stimuli. The second light sensor 122 may receive a plurality of parameters (for example, color temperature, illuminance, color coordinates, and color rendering circadian illuminance associated with the eyes of the user U) of the parameters A, B, and C associated with the eyes of the user U and the corresponding circadian stimuli. In this way, the first output light parameter from the table top K and the second output light parameter received by the eyes of the user U may be received and analyzed.

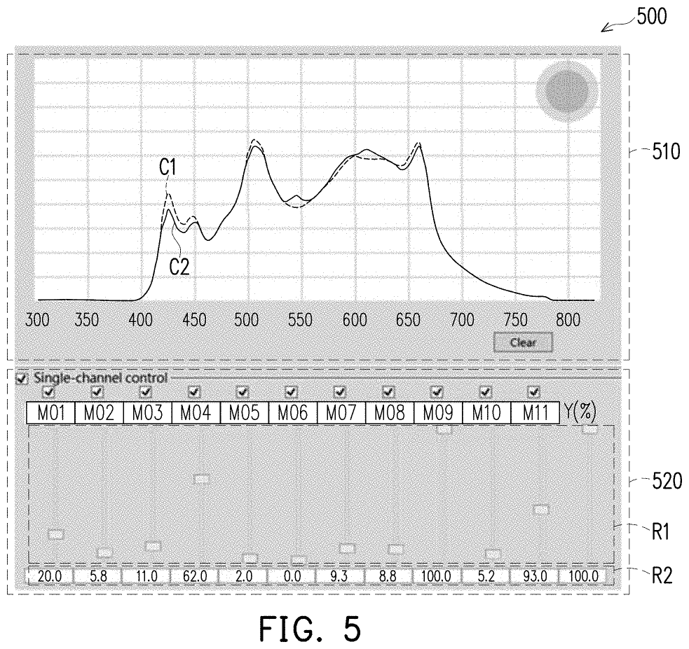

Referring to FIGS. 1 and 5, FIG. 5 illustrates a schematic diagram of an operational interface according to an embodiment of the disclosure. An operational interface 500 may be provided by the operating module 130. For example, the light supply system 100 includes a display. The operating module 130 may provide the operational interface 500 and control the display to display the operational interface 500. In this embodiment, the operational interface 500 displays the relevant information of the first light parameter P1 of the first light L1. For example, a zone 510 of the operational interface 500 displays a predetermined spectrum C1 and a measured spectrum C2. The predetermined spectrum C1 is the spectrum of the first light L1 that is previously stored. The measured spectrum C2 is the spectrum of the first light L1 currently measured by the light sensing device 120.

In this embodiment, a zone 520 of the operational interface 500 displays the light output of the light emitting modules M01 to M11 corresponding to the measured spectrum C2. For example, the zone 520 includes subzones R1 and a R2. The subzone R2 displays the light output of the light emitting modules M01 to M11 in the form of number values. The subzone R1 displays the light output of the light emitting modules M01 to M11 by the high and low position of a plurality of icons.

In this embodiment, the columns labeled “M01” to “M11” are respectively used to display the light output status of the light emitting modules M01 to M11. The larger the number value or the higher the position of an icon, the larger the output of the corresponding light emitting module. The smaller the number value or the lower the position of an icon, the smaller the output of the corresponding light emitting module. For example, the position of an icon reflects the size of the number value. A number value of 0 means zero output. A number value of 100 means a maximum output. In addition, the column labeled “Y(%)” is used to display the overall light output of the light source device 110. The overall light output is based on the light output ratio of the light emitting modules M01 to M11.

In this embodiment, when the measured spectrum C2 is determined to deviate from the predetermined spectrum C1 by greater than a predetermined value, it is indicated that the first light L1 provided by the light source device 110 has deviated. For example, it is indicated that the luminous performance of at least one light emitting device in the light emitting modules M01 to M11 is degraded or damaged, or that light other than the first light L1 is generated in the care setting. The monitoring module 130 may adjust the light output ratio of the light emitting modules M01 to M11 so that the measured spectrum C2 is close to the expected predetermined spectrum C1. In other words, the light supply system 100 may adjust the first light parameter P1 through the operational interface 500. Once the light output ratio or the overall light output of the light emitting modules M01 to M11 is changed, the monitoring module 130 provides the corresponding control signal SC in real time. On the other hand, when the measured spectrum C2 is determined to deviate from the predetermined spectrum C1 by less than or equal to the predetermined value, it is indicated that the measured spectrum C2 is similar to the predetermined spectrum C1. Therefore, the monitoring module 130 does not adjust the light output ratio of the light emitting modules M01 to M11.

In this embodiment, the operational interface 500 further displays the color temperature and illuminance generated by the first light L1. In some embodiments, the operational interface 500 displays the color temperature, illuminance, color rendering index (CRI), and color deviation (Duv) generated by the first light L1. In some embodiments, the light supply system 100 may adjust the light output ratio of the light emitting modules M01 to M11 to optimize at least one of the illuminance, luminous range, color rendering index, and chromatic aberration of the first light L1. In this way, the high color rendering performance of the first light L1 may be maintained. For example, the light supply system 100 sets the main optimization objectives to be the spectrum and illuminance of the first light L1, and the secondary optimization objectives to be the luminous range, color rendering, and chromatic aberration of the first light L1 (the disclosure is not limited thereto).

It is worth mentioning that the half-height widths of the spectra of the light emitting modules M01 to M11 are all less than 30 nanometers. Therefore, the light supply system 100 may monitor the light parameters and adjust the light output ratio of the light emitting modules M01 to M11, so that the light source device 110 may maintain high color rendering performance.

In this embodiment, an operator may adjust the light output of the light emitting modules M01 to M11 by dragging the position of at least one of the icons in the subzone R1 of the zone 520 up or down through touch or mouse operation. In this embodiment, the operator may also adjust the light output of light emitting modules M01 to M11 by entering the number value in the subzone R2 of the zone 520.

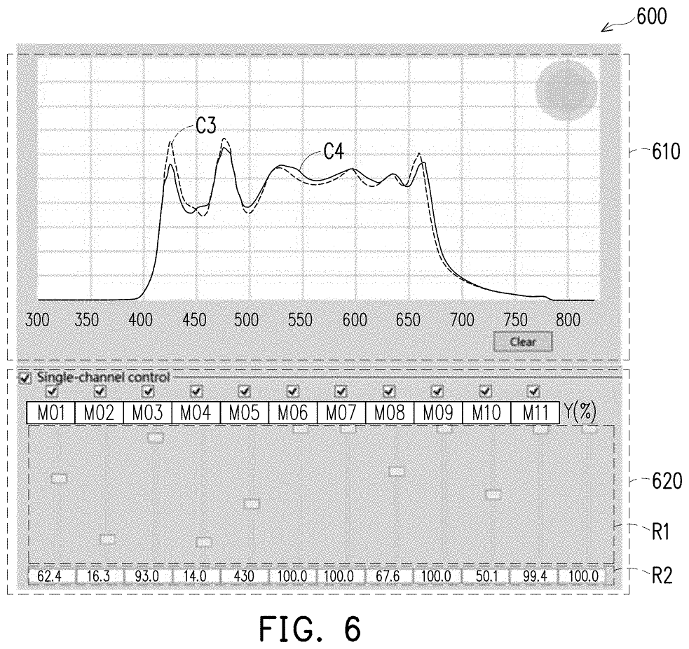

Referring to FIGS. 1 and 6, FIG. 6 illustrates a schematic diagram of an operational interface according to another embodiment of the disclosure. An operational interface 600 may be provided by the operating module 130. In this embodiment, the operational interface 600 displays the relevant information of the second light parameter P2 of the second light L2. For example, similar to the operational interface 500, a zone 610 of the operational interface 600 displays a predetermined spectrum C3 and a measured spectrum C4. The predetermined spectrum C3 is the spectrum of the second light L2 that is previously stored. The measured spectrum C4 is the spectrum of the second light L2 currently measured by the light sensing device 120. In this embodiment, a zone 620 of the operational interface 600 displays the light output of the light emitting modules M01 to M11 corresponding to the measured spectrum C4. For example, the zone 620 includes the subzones R1 and R2. The subzone R2 displays the light output of the light emitting modules M01 to M11 in the form of number values. The subzone R1 displays the light output of the light emitting modules M01 to M11 by the high and low position of a plurality of icons.

In this embodiment, when the measured spectrum C4 is determined to deviate from the predetermined spectrum C3 by more than a predetermined value, it is indicated that that the second light L2 provided by the light source device 110 has deviated. For example, it is indicated that the luminous performance of at least one light emitting device in the light emitting modules M01 to M11 is degraded or damaged, or that light other than the second light L2 is generated in the care setting. The monitoring module 130 may adjust the light output ratio of the light emitting modules M01 to M11 so that the measured spectrum C4 is close to the expected predetermined spectrum C3. In other words, the light supply system 100 may adjust the second light parameter P2 through the operational interface 600. On the other hand, when the measured spectrum C4 is determined to deviate from the predetermined spectrum C3 less than or equal to the predetermined value, it is indicated that the measured spectrum C4 is similar to the predetermined spectrum C3. Therefore, the monitoring module 130 does not adjust the light output ratio of the light emitting modules M01 to M11.

In some embodiments, the operational interface 600 displays the color temperature, illuminance, color rendering index (CRI) and color deviation (Duv) generated by the second light L2. In some embodiments, the light supply system 100 may adjust the light output ratio of the light emitting modules M01 to M11 to optimize at least one of the luminous range, color rendering index, and chromatic aberration of the second light L2. In this way, the high color rendering performance of the second light L2 may be maintained. For example, the light supply system 100 sets the main optimization objectives to be the spectrum and illuminance of the spectrum of second light L2, and the secondary optimization objectives to be the luminous range, color rendering, and chromatic aberration of the second light L2 (the disclosure is not limited thereto). In this embodiment, once the light output ratio or the overall light output of the light emitting modules M01 to M11 is changed, the monitoring module 130 provides the corresponding control signal SC in real time.

Similar to the operational interface 500, an operator may adjust the light output of the light emitting modules M01 to M11 by dragging the position of at least one of the icons in the subzone R1 of the subzone 620 up or down through touch or mouse operation. In this embodiment, the operator may also adjust the light output of light emitting modules M01 to M11 by entering the number value in the subzone R2 of the zone 620.

In summary, a plurality of light emitting modules of the light source device of the disclosure have different central wavelengths, and the half-height width of a plurality of spectra of the light emitting modules is lower than 30 nanometers. Thus, in the disclosure, the different ratios of each band of the spectra may be adjusted through the accurate light sensing device and monitoring method, so that the light emitting modules may accurately restore the second light parameter under different light source parameters. Therefore, the light supply method and the light supply system of the disclosure may accurately provide the second light with the second light parameter, so that the physiology of the user may be improved. In addition, the disclosure may further adjust the light output ratio between the light emitting modules to optimize at least one of the spectrum, illuminance, illuminous scope, color rendering, and chromatic aberration of the second light, so as to maintain the second light at the second light parameter.

Although the disclosure has been disclosed in the above by way of embodiments, the embodiments are not intended to limit the disclosure. Those with ordinary knowledge in the technical field can make various changes and modifications without departing from the spirit and scope of the disclosure. Therefore, the protection scope of the disclosure is subject to the scope of the appended claims.

What is claimed is:

1. A light supply method for phototherapy, comprising:
driving a plurality of light emitting modules of a light source device so that the light source device outputs a first light;
sensing the first light by a light sensing device to receive a first light parameter of the first light;
receiving a second light parameter corresponding to best physiology of a user; and
adjusting a light output ratio of the light emitting modules based on the second light parameter and adjusting the first light parameter to the second light parameter, thereby adjusting the first light to a second light,
wherein the light emitting modules respectively have a different central wavelength, wherein a half-height width of a plurality of spectra of the light emitting modules is less than 30 nanometers.

2. The light supply method according to claim 1, wherein adjusting the light output ratio of the light emitting modules comprises:
adjusting the light output ratio of the light emitting modules to optimize at least one of illuminance, luminous range, color rendering index, and chromatic aberration of at least one of the first light and the second light.

3. The light supply method according to claim 1, wherein:
the light sensing device comprises a first light sensor;
the first light sensor and the light source device are separated by a first predetermined distance; and
the light supply method further comprises:
based on the first predetermined distance, receiving the second light by the first light sensor to receive a first output light parameter.

4. The light supply method according to claim 3, wherein:
the light sensing device further comprises a second light sensor;
the second light sensor and the first light sensor are separated by a second predetermined distance; and
the light supply method further comprises:
based on the second predetermined distance, receiving light reflected from a table top by the second light sensor to receive a second output light parameter.

5. The light supply method according to claim 3, further comprising:
controlling illuminance of the second light provided to the first light sensor to be between 5800 lux and 6200 lux; and
controlling a color temperature of the second light to be between 4800 k and 5200 k.

6. The light supply method according to claim 3, further comprising:
controlling illuminance of the second light provided to the first light sensor to be between 2800 lux and 3200 lux; and
controlling a color temperature of the second light to be one of between 4800 k and 5200 k and between 7800 k and 8200 k.

7. The light supply method according to claim 1, wherein the best physiology is best data or target data associated with at least one of sleep quality, inhibition condition of melatonin during the daytime, and a depression scale analysis of the user.

8. The light supply method according to claim 1, further comprising:
providing a monitor interface, and adjusting at least one of the first light parameter and the second light parameter through the monitor interface.

9. The light supply method according to claim 1, wherein:
a first central wavelength of a first light emitting module among the light emitting modules is adjacent to a second central wavelength of a second light emitting module, and
a wavelength difference between the first central wavelength and the second central wavelength is less than or equal to 40 nanometers.

10. A light supply system for phototherapy, comprising:
a light source device, comprising a plurality of light emitting modules, wherein the light emitting modules are driven to provide a first light;
a light sensing device, configured to sense the first light to receive a first light parameter of the first light; and
a monitoring module, coupled to the light source device and the light sensing device, configured to receive a second light parameter corresponding to best physiology of a user and to adjust a light output ratio of the light emitting modules based on the second light parameter and adjust the first light parameter to the second light parameter, thereby adjusting the first light to a second light,
wherein the light emitting modules respectively have a different central wavelength, and a half-height width of a plurality of spectra of the light emitting modules is less than 30 nanometers.
